(19) United States
(12) Patent Application Publication
(10) Pub. No.: US 2025/0040899 A1
(43) Pub. Date: Feb. 6, 2025

(54) MOBILE RADIOGRAPHIC IMAGING SYSTEM

(71) Applicant: KONICA MINOLTA, INC., Tokyo (JP)

(72) Inventors: Kojiro ONO, Tokyo (JP); Miyako SUGIHARA, Tokyo (JP); Takanori KAKIGI, Tokyo (JP); Takeshi MURAYAMA, Tokyo (JP)

(73) Assignee: KONICA MINOLTA, INC., Tokyo (JP)

(21) Appl. No.: 18/786,102

(22) Filed: Jul. 26, 2024

(30) Foreign Application Priority Data
Jul. 31, 2023 (JP) 2023-124875

(51) Int. Cl. A61B 6/00; A61B 6/10; A61B 6/40; A61B 6/46

(52) U.S. Cl. CPC A61B 6/4405; A61B 6/107; A61B 6/405; A61B 6/4417; A61B 6/461; A61B 6/487; A61B 6/542

(57) ABSTRACT

Provided is a mobile radiographic imaging system that includes: a radiation generating apparatus that emits radiation; and a control means capable of setting the radiation generating apparatus to a plurality of modes including a first mode, in which the radiation generating apparatus performs moving image imaging with an allowable radiation dose, and a second mode, in which the radiation generating apparatus performs the moving image imaging with an allowable radiation dose smaller than the allowable radiation dose in the first mode.

### CROSS REFERENCE TO RELATED APPLICATIONS

The entire disclosure of Japanese Patent Application No. 2023-124875, filed on Jul. 31, 2023, is incorporated herein by reference in its entirety.

### BACKGROUND

### Technological Field

The present disclosure relates to a mobile radiographic imaging system.

### Description of Related Art

A fluoroscopic apparatus that continuously emits pulsed radiation to perform fluoroscopic imaging of a subject has been proposed (e.g., Japanese Patent Publication Laid-Open No. 2021-53168). The dose of radiation emitted in the fluoroscopic imaging is more than in the general imaging.

According to the Japanese laws and regulations, a fluoroscopic apparatus is required to be used in a strictly controlled area, and cannot be used in a general hospital room. Therefore, when fluoroscopic imaging is performed, the patient needs to move to an imaging room in which a fluoroscopic apparatus can be used, and in the case of a patient who is difficult to move to the imaging room, such as a critically ill patient or a patient with an infectious disease, the physical burden on the patient is large. In addition, when a critically patient or a patient with an infectious disease is moved, it is necessary to assist the patient in his/her movement or to construct an environment for a countermeasure against infection, which increases the physical burden and the time burden on a healthcare professional.

The above-described problems can be solved if hospitals possess a mobile dynamic imaging apparatus (for example, Japanese Patent Publication Laid-Open No. 2022-11259) capable of imaging moving images even in a general hospital room. However, it is a large financial burden for hospitals to possess each of a fluoroscopic apparatus and a mobile dynamic imaging apparatus, and in particular it is difficult for small-scale hospitals and clinics to possess both a fluoroscopic apparatus and a mobile dynamic imaging apparatus.

### SUMMARY

An object of the present disclosure is to provide a mobile radiographic imaging system capable of reducing a burden on a subject or a healthcare professional.

The present inventors have considered that the above-described problems can be solved by providing a mobile radiographic imaging system that has a plurality of moving image imaging modes including an imaging mode, in which fluoroscopic imaging is performed, and a dynamic imaging mode, which is a general imaging mode in which a series of images of a plurality of frames are acquired by irradiating a subject with radiation. However, the present inventors have found that there is a new problem in that a wrong radiation exposure may occur if the radiation dose is not set more accurately in the fluoroscopic imaging and the moving image imaging in the general imaging, in the control when moving image imaging is performed.

A mobile radiographic imaging system in an embodiment of the present disclosure includes: a radiation generating apparatus that emits radiation; and a setting means capable of setting the radiation generating apparatus to a plurality of modes including a first mode, in which the radiation generating apparatus performs moving image imaging with an allowable radiation dose, and a second mode, in which the radiation generating apparatus performs the moving image imaging with an allowable radiation dose smaller than the allowable radiation dose in the first mode.

A mobile radiographic imaging system in an embodiment of the present disclosure includes: a radiation generating apparatus that emits radiation; a setting means that switches between a first mode, in which the radiation generating apparatus is used as a fluoroscopic apparatus, and a second mode, in which the radiation generating apparatus is used as a general imaging apparatus; and an input means that inputs a setting of the radiation generating apparatus to the setting means, and in a case where the second mode is set by the setting means, the input means is prohibited from inputting a setting for an allowable radiation dose that exceeds the allowable radiation dose in the second mode.

Note that, these generic or specific aspects may be implemented as a system, an apparatus, a method, an integrated circuit, a computer program, or a recording medium, or any selective combination of a system, an apparatus, a method, an integrated circuit, a computer program, and a recording medium.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a diagram illustrating an example of a medical cart in use;

FIG. 2 is a diagram illustrating an example of the medical cart while moving or not in use;

FIG. 3 is a diagram illustrating functional blocks of the medical cart; and

FIG. 4 is a diagram illustrating an example of a flowchart of processing performed by a setting means.

### DETAILED DESCRIPTION OF EMBODIMENTS

Hereinafter, one or more embodiments of the present invention will be described with reference to the drawings. However, the scope of the invention is not limited to the disclosed embodiments.

Hereinafter, an embodiment of the present disclosure will be described in detail with reference to the accompanying drawings as appropriate.

### <1. Mobile Radiographic Imaging System>

First, a schematic configuration of a mobile radiographic imaging system according to an embodiment of the present disclosure will be described. The mobile radiographic imaging system includes at least a radiation generating apparatus. The mobile radiographic imaging system may be, for example, medical cart 100 that is movable. Medical cart 100 may have an automatic driving function.

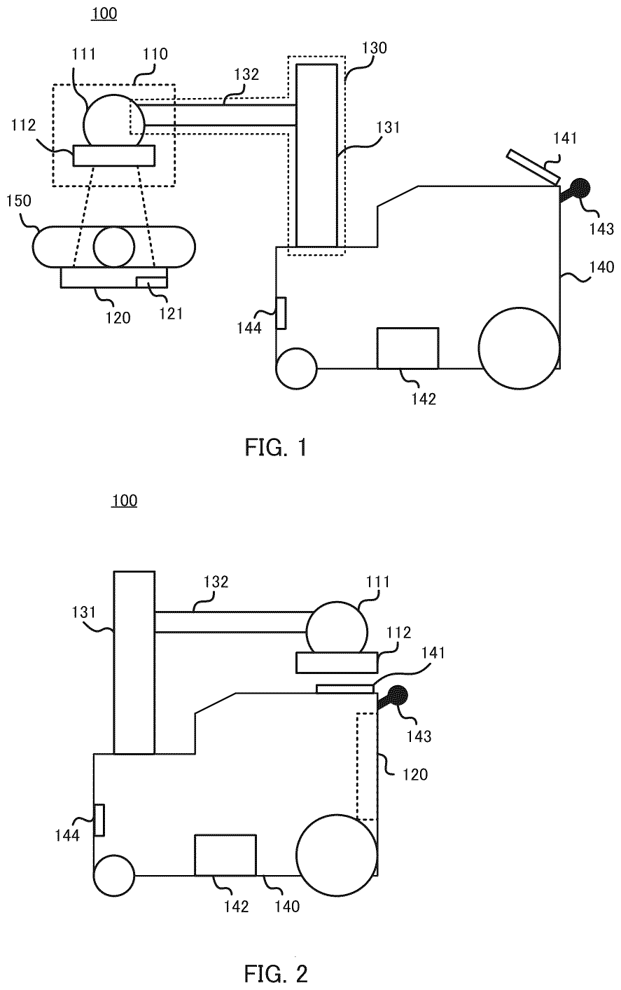

FIG. 1 is a diagram illustrating an example of medical cart 100 in use.

FIG. 2 is a diagram illustrating an example of medical cart 100 while moving or not in use.

Medical cart 100 includes radiation generating apparatus 110, radiation detection apparatus 120, arm 130, and main body 140. Medical cart 100 can be set to, as the imaging mode, at least a fluoroscopy mode as a first mode or a dynamic imaging mode as a second mode, can be used as a fluoroscopic apparatus in a case where the imaging mode is set to the fluoroscopy mode, and can be used as a dynamic imaging apparatus in a case where the imaging mode is set to the dynamic imaging mode. Medical cart 100 may have three or more imaging modes.

Medical cart 100 may be communicable with a hospital information system (HIS; not illustrated), a radiology information system (RIS; not illustrated), an image analysis apparatus (not illustrated), a picture archiving and communication system (PACS; not illustrated), other radiography apparatuses (not illustrates), and/or the like.

### [Radiation Generating Apparatus]

Radiation generating apparatus 110 includes generator 111 and collimator 112. Radiation generating apparatus 110 may be a radiation generating unit.

Generator 111 includes a tube. When a voltage is applied to the tube and a current flows, radiation is emitted from the tube. A pulsed voltage may be applied to the tube, and pulsed radiation, for example, X-rays may be emitted from the tube.

The maximum radiation dose that generator 111 emits, that is, the allowable radiation dose, is limited according to the imaging mode set to medical cart 100. In the dynamic imaging mode, the radiation dose to be emitted is limited due to the limitation as a general imaging apparatus. In the fluoroscopy mode, there is no limitation as a general imaging apparatus, and thus, a larger radiation dose can be emitted than in the dynamic imaging mode. In the fluoroscopy mode, the radiation dose may not be limited. Further, generator 111 emits radiation on the basis of a setting(s) through an input section. The imaging conditions are, for example, conditions related to subject 150, such as an imaging portion, an imaging direction, and a physique, and conditions related to radiation emission, such as a tube voltage, a tube current, an irradiation time, a current-time product (mAs value), a frame rate, the number of allowable frames, a pulse width, a pulse interval, a pulse cycle, and a pulse duty ratio.

In the dynamic imaging mode, radiation generating apparatus 110 repeatedly emits radiation pulses for a predetermined time (duration) at a cycle of a plurality of times per unit time (for example, fifteen times per second) while radiation emission is instructed. Here, the pulse cycle and the duration are set in advance by input/output section 141. The pulse interval, the pulse width, and the pulse duty ratio may be set in advance by input/output section 141. Note that, although a configuration in which a radiation pulse is emitted in the dynamic imaging mode in the present embodiment will be described as an example, a configuration in which radiation is continuously emitted may be employed. In the dynamic imaging mode, radiation emission is limited due to the limitation as a general imaging apparatus.

In the fluoroscopy mode, radiation generating apparatus 110 repeats radiation pulses at a cycle of a plurality of times per unit time (e.g., six times, ten times, or fifteen times per second) while radiation emission is instructed. Here, the pulse period is set in advance by input/output section 141. In the fluoroscopy mode, no duration is set. That is, in the fluoroscopy mode, radiation emission is not limited by the duration. Further, in the fluoroscopy mode, there is no limitation as a general imaging apparatus, and thus, more radiation can be emitted than in the dynamic imaging mode. In the fluoroscopy mode, the radiation dose may not be limited. The pulse interval, the pulse width, and the pulse duty ratio may be set in advance by input/output section 141. Note that, although a configuration in which a radiation pulse is emitted in the fluoroscopy mode in the present embodiment will be described as an example, a configuration in which radiation is continuously emitted may be employed.

Collimator 112 narrows an irradiation field of radiation to be emitted. Collimator 112 may include a shielding means such as a filter. The shielding means may narrow an area to be irradiated with radiation to be emitted. The shielding means may reduce the intensity of radiation to be emitted.

### [Radiation Detection Apparatus]

Radiation detection apparatus 120 includes communication section 121. Further, although not illustrated, radiation detection apparatus 120 includes a sensor board, a scanning circuit, a readout circuit, a control section, and the like. Radiation detection apparatus 120 may be a radiation detection unit.

Radiation detection apparatus 120 detects radiation emitted from radiation generating apparatus 110 via subject 150. Subject 150 is, for example, a human, an animal, or the like.

In the sensor board, pixels are arranged two-dimensionally (in a matrix). Each pixel includes a radiation detection element, which generates an electric charge corresponding to the dose of radiation received via the subject, and a switch element, which accumulates and releases the electric charge. The scanning circuit turns on or off each switch element. The readout circuit reads out the amount of charge released from each pixel as a signal value (intensity). The control section controls radiation detection apparatus 120 in its entirety. The control section generates a radiation image from a plurality of signal values read by the reading circuit. Communication section 121 transmits radiation image data, various signals, and the like generated by the readout circuit to the outside. Further, communication section 121 receives various pieces of information and various signals. Communication section 121 may perform wireless communication or may perform wired communication.

In each pixel of radiation detection apparatus 120 configured in such a manner, when the radiation detection element receives radiation in a state in which the scanning circuit turns off the switch element, the radiation detection element generates an electric charge corresponding to the dose of radiation and accumulates the electric charge. When the scanning circuit turns on the switch element, the accumulated charge is released, and the readout circuit detects the amount of charge released from each pixel and generates a signal value indicating the amount of charge. The control section generates a radiation image based on the signal value generated for each pixel. The generated individual radiation images are one still image in the dynamic imaging. In the case of pulsed radiation, one still image is generated for each pulse.

Communication section 121 performs communication with communication section 144 of main body 140 and transmits a generated radiation image to communication section 144 of main body 140. Communication section 121 may transmit a still image to main body 140 each time one still image is generated, or may collectively transmit a plurality of still images to communication section 144 of main body 140. Communication section 121 may communicate with a component other than main body 140. The communication performed by communication section 121 may be wireless communication or wired communication.

In a case where radiation generating apparatus 110 performs pulse emission, timings at which a plurality of still images constituting a dynamic image is generated are synchronized with timings at which radiation is emitted from radiation generating apparatus 110. In a case where radiation generating apparatus 110 performs continuous emission, on the other hand, generation of a plurality of still images constituting a dynamic image is performed at arbitrary timings during the time of the continuous emission.

Radiation detection apparatus 120 may be stored in a storage section provided in main body 140 when medical cart 100 moves or is not in use. When radiation detection apparatus 120 is stored in the storage section of main body 140, radiation detection apparatus 120 may be connected to main body 140 in a wired manner, and radiation detection apparatus 120 may be charged from main body 140 and may perform communication with main body 140. When radiation detection apparatus 120 is stored in the storage section of main body 140, software or firmware of radiation detection apparatus 120 may be updated by communication with main body 140. Radiation detection apparatus 120 may be charged and performs communication with main body 140 wirelessly.

Radiation detection apparatus 120 may be wirelessly connected during use. Radiation detection apparatus 120 may be driven by an internal battery. Power may be supplied to radiation detection apparatus 120 from main body 140 in a wired or wireless manner.

Radiation detection apparatus 120 may be connected to main body 140 in a wired manner when moving or not being in use, and may be connected to main body 140 in a wireless manner when being in use.

### [Arm]

Arm 130 includes vertical arm 131 and horizontal arm 132. Vertical arm 131 pivotably supports horizontal arm 132, that is, in a rotatable manner with respect to the Z-axis. Furthermore, vertical arm 131 may movably support horizontal arm 132. Horizontal arm 132 rotatably supports radiation generating apparatus 110, that is, in a rotatable manner with respect to the X-axis and the Y-axis. Radiation generating apparatus 110 can be set in any orientation with respect to main body 140 by vertical arm 131 and horizontal arm 132. The position and orientation of the arm may be determined by an input through input/output section 141. Vertical arm 131 and horizontal arm 132 may be rotatable and movable by the control by main body 140 or may be rotatable and movable manually.

### [Main Body]

Main body 140 includes input/output section 141, power supply section 142, and communication section 144. Input/output section 141 may be an input/output apparatus outside main body 140. Main body 140 may include handle 143. Although not illustrated, main body 140 may include a storage section that stores radiation detection apparatus 120. The communication between communication section 144 of main body 140 and communication section 121 of radiation detection apparatus 120 may be wireless communication or wired communication.

Although not illustrated, main body 140 may include a means for locking the movement. By releasing the means for locking the movement, main body 140 can be moved. The means for locking the movement may be means for wheel fixing.

Input/output section 141 can have a variable inclination angle with respect to main body 140. Input/output section 141 may be separable from main body 140. In a case where input/output section 141 is separated from main body 140, input/output section 141 and main body 140 are connected to each other in a wireless or wired manner.

Input/output section 141 is constituted by an input means and an output means. In input/output section 141, an input section and an output section may be separated. Input/output section 141 may be constituted by a touch screen, a keyboard, a mouse, a microphone, a camera, a display, a speaker, a display lamp, and/or the like. For input/output section 141, input means for an input by voice, an input by gesture, an input by line of sight, an input by brain waves, and/or the like may be available. Input/output section 141 performs settings for radiation generating apparatus 110. Further, input/output section 141 allows an operation for instructing radiation generating apparatus 110 to emit radiation to be inputted. Input/output section 141 may configure an emission instruction switch with an emission button of a toggle button and output a radiation emission instruction during a period from when the toggle button is pressed for the first time to when the toggle button is pressed for the second time, or may output a radiation emission instruction during a period in which the emission button is continuously pressed.

With an operation on input/output section 141, generator 111 emits radiation of a dose set by input/output section 141. The radiation is, for example, X-rays.

Various parameters for radiation emission by radiation generating apparatus 110 are inputted to the input section. The input section may receive an input of mode switching. In a case where medical cart 100 is set to the dynamic imaging mode by setting means 330, the input section may be prohibited from inputting a setting for an allowable radiation dose that exceeds the allowable radiation dose in the dynamic imaging mode.

The output section performs a notification regarding mode switching. The output section may perform a notification prompting switching from the fluoroscopy mode to the dynamic imaging mode. The output section may perform a notification regarding the current mode of medical cart 100. The output section may perform a notification that switching from the fluoroscopy mode to the dynamic imaging mode has been performed. The output section may issue a warning. Note that, the notification will be described in detail later.

Power supply section 142 supplies power to main body 140 in its entirety. Power supply section 142 may charge radiation detection apparatus 120. Power supply section 142 may be constituted by a rechargeable battery. Power supply section 142 may be capable of performing charging by being connected to a commercial power supply or the like. Power supply section 142 may have a function of detecting that charging has started.

Power supply section 142 may be, for example, a nickel-metal hydride battery, a lithium-ion battery, a sodium battery, an all-solid-state battery, a flywheel, or the like. Power supply section 142 may be constituted by a fuel cell or a nuclear battery. The fuel cell may be refillable with fuel.

Power supply section 142 may be connected, when not in use, to a commercial power supply or the like to perform charging, and may be disconnected, when in use, from the commercial power supply to perform power supply by a battery. When power supply section 142 is not in use, the fuel of the fuel cell may be filled.

Handle 143 is held by a person in a case where medical cart 100 is moved. A sensor built in handle 143 may detect that handle 143 is held by a person. The sensor may notify setting means 330 when the sensor detects that handle 143 is held by a person.

Communication section 144 performs communication with communication section 121 of radiation detection apparatus 120. Communication section 144 transmits settings and instructions set by input/output section 141 to communication section 121 of radiation detection apparatus 120. Further, communication section 144 outputs a radiation image received from communication section 121 of radiation detection apparatus 120 to input/output section 141.

### [Functional Blocks]

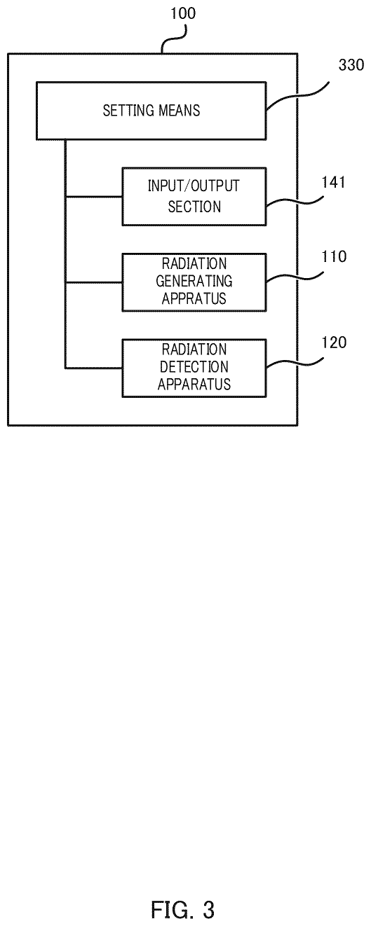

FIG. 3 is a diagram illustrating functional blocks of medical cart 100. Medical cart 100 includes input/output section 141, radiation generating apparatus 110, and radiation detection apparatus 120.

Setting means 330 controls medical cart 100 in its entirety. Setting means 330 may be present anywhere in medical cart 100, but is preferably present in main body 140. Setting means 330 is constituted by a computer such as a central processing unit (CPU), an application specific integrated circuit (ASIC), and a field programmable gate array (FPGA). Setting means 330 can manage the modes of medical cart 100 and switch between the modes. Setting means 330 can at least select and switch between the fluoroscopy mode used as a fluoroscopic apparatus or the general imaging mode used as a general imaging apparatus. Setting means 330 may be capable of selecting an imaging mode from three or more modes including any other mode. Hereinafter, the mode used as a general imaging apparatus will be referred to as the general imaging mode. The general imaging mode may be the dynamic imaging mode used as a dynamic imaging apparatus. Setting means 330 may rotate and move vertical arm 131 and horizontal arm 132.

Mode setting information and setting information on radiation generating apparatus 110, that is, imaging conditions are inputted to setting means 330 from input/output section 141. In the case of the general imaging mode, setting means 330 may cause input/output section 141 to reject an input whereby an imaging condition for an allowable radiation dose that exceeds the allowable radiation dose for the general imaging apparatus is about to be inputted. In a case where the inputted setting information is a condition that cannot be allowed as the general imaging mode, setting means 330 may cause input/output section 141 to perform a notification.

The mode setting information and the setting information on radiation generating apparatus 110 are inputted to the input means. The output means may display the inputted setting information, or may perform a notification in a case where setting information that is not required as the general imaging mode is inputted. Various parameters for radiation emission by radiation generating apparatus 110 are inputted to the input means. The input means may receive an input of mode switching. In a case where medical cart 100 is set to the dynamic imaging mode by setting means 330, the input section may be prohibited from inputting a setting for an allowable radiation dose that exceeds the allowable radiation dose in the dynamic imaging mode.

Radiation generating apparatus 110 emits radiation such as X-rays under the control of setting means 330.

Radiation detection apparatus 120 detects radiation emitted by radiation generating apparatus 110. Radiation detection apparatus 120 transmits information on the detected radiation to main body 140.

### [Operation]

In the dynamic imaging mode, radiation generating apparatus 110 repeats, based on the control of setting means 330, emission of pulsed radiation a plurality of times per predetermined time (for example, fifteen times per second) until the duration is reached.

In a case where radiation generating apparatus 110 cannot repeat the emission of pulsed radiation a plurality of times per predetermined time, radiation generating apparatus 110 may continuously emit radiation for a predetermined time. The phrase “emit radiation for a predetermined time” in the present disclosure includes both pulsed emission in which emission of pulsed radiation is repeated a plurality of times over a predetermined time and continuous emission in which radiation is continuously emitted for a predetermined time. The duration is, for example, fifteen seconds. In the dynamic imaging mode, when radiation emission is instructed by the emission instruction switch, radiation is emitted with the duration as the maximum irradiation time. That is, in the dynamic imaging mode, radiation is emitted within the duration and in a case where the emission instruction switch is operated.

The intensity of radiation in the dynamic imaging mode may be lower than the intensity of radiation in the fluoroscopy mode.

In the dynamic imaging mode, the radiation dose of radiation to be emitted in one pulse may be fewer than the dose of radiation emitted in a case where a still image is imaged, and thus, the dose of radiation as a whole with which a patient is irradiated can be made equivalent to that in a case where a still image of the chest is imaged. Note that, in the case of amount of exposure of a patient.

In the fluoroscopy mode, the imaging conditions of radiation generating apparatus 110 are set by input/output section 141 under the control of setting means 330 such that the emission of pulsed radiation is repeated a plurality of times per predetermined time (for example, fifteen times per second) based on the operation of the emission button. In the fluoroscopy mode, the duration may not be set. In the fluoroscopy mode, a longer duration may be set than in the dynamic imaging mode.

In a case where radiation generating apparatus 110 cannot repeat the emission of pulsed radiation a plurality of times per predetermined time, radiation generating apparatus 110 may continuously emit radiation for a predetermined time. In the fluoroscopy mode, radiation is emitted while radiation emission is instructed by the emission button. That is, in the case where no duration is set in the fluoroscopy mode, radiation is emitted without any time limitation while radiation emission is instructed.

Setting means 330 sets various imaging conditions (a tube voltage, a tube current, a tube current-time product (mAs value), an imaging portion, an imaging direction, and the like) to at least radiation generating apparatus 110 based on an input through input/output section 141. Setting means 330 may set various imaging conditions to radiation detection apparatus 120. Setting means 330 may set various imaging conditions based on imaging order information acquired from another system (HIS, RIS, or the like) or a user (for example, a technician). Setting means 330 may set various imaging conditions to radiation generating apparatus 110 and/or radiation detection apparatus 120, for example, based on the imaging order information or a table in which users and the various imaging conditions are associated with each other.

Setting means 330 may cause input/output section 141 to issue a warning in a case where the set various imaging conditions do not satisfy the use as the general imaging apparatus and in a case where the current mode notified from setting means 330 is the general imaging mode.

### [Mode]

Medical cart 100 has a plurality of modes, for example, a general imaging mode and the fluoroscopy mode. Medical cart 100 may have three or more modes. The general imaging mode may be the dynamic imaging mode. An imaging mode may be inputted through input/output section 141 and mode switching may be performed. The mode may be switched by being selected from a plurality of imaging modes displayed on input/output section 141. The mode may be switched by a switch. The imaging mode of medical cart 100 may be switched according to a patient to be imaged, an examination order, or an imaging order.

The radiation dose of radiation to be emitted in the dynamic imaging mode may be different from the radiation dose of radiation to be emitted in the fluoroscopy mode. The radiation dose of radiation to be emitted in the fluoroscopy mode may be larger than the radiation dose of radiation to be emitted in the dynamic imaging mode.

Further, in the dynamic imaging mode, there is an irradiation time, whereas in the fluoroscopy mode, there is no irradiation time. That is, medical cart 100 no longer emits radiation after the irradiation time elapses even when medical cart 100 continues to output a radiation emission instruction in the dynamic imaging mode, whereas medical cart 100 emits radiation all the time while medical cart 100 is outputting a radiation emission instruction in the fluoroscopy mode.

In a case where the fluoroscopy mode is set, setting means 330 may cause input/output section 141 to display a moving image generated by radiation detection apparatus 120 in real time. In a case where the dynamic imaging mode is set, setting means 330 may cause input/output section 141 to display a moving image that is more limited than a moving image displayed in a case where the fluoroscopy mode is set. In a case where the dynamic imaging mode is set, input/output section 141 may display, for example, a moving image delayed relative to that in a case where the fluoroscopy mode is set. In a case where the dynamic imaging mode is set, input/output section 141 may display, for example, a moving image with a frame rate smaller than that in a case where the fluoroscopy mode is set. Setting means 330 may limit a moving image generated by radiation detection apparatus 120 or may cause radiation detection apparatus 120 to limit a moving image to be generated.

When setting means 330 causes input/output section 141 to display a moving image corresponding to the set mode, the imaging apparatus can be used in a restricted area in the same manner as a fluoroscopic apparatus and can be used as a dynamic imaging apparatus in a general hospital room or the like.

### [Limitation of Radiation Dose]

Setting means 330 causes radiation emission to stop in a case where an allowable radiation dose has been emitted. Radiation generating apparatus 110 stops emitting radiation in a case where an allowable radiation dose has been emitted.

Setting means 330 sets parameters for radiation generating apparatus 110 to emit radiation. The radiation dose of radiation to be emitted from radiation generating apparatus 110 in the general imaging mode may be fewer than the radiation dose of radiation to be emitted in the fluoroscopy mode.

The allowable radiation dose is determined according to the set mode. The allowable radiation dose in the dynamic imaging mode is fewer than the allowable radiation dose in the fluoroscopy mode. The radiation dose can be limited by limiting the radiation emission time, limiting the intensity of radiation, and/or limiting an area to be irradiated with radiation. The limitation of the radiation emission time can be performed by control to change the entire imaging time or control not to change the entire imaging time. The control not to change the entire imaging time can be performed by limiting the frame rate and/or by limiting the pulse duty ratio. The pulse duty ratio can be limited by limiting the pulse width and/or by limiting the pulse interval. The intensity of radiation can be limited by control of a tube and/or control using a shielding object, that is, a filter. The tube can be controlled by control of the tube current, control of the tube voltage, and/or control of the tube current-time product. The area to be irradiated with radiation can be limited by shielding a part of the emitted radiation with a shielding object.

Setting means 330 may limit the irradiation time by limiting the entire imaging time. The entire imaging time can be limited by setting the allowable time. In radiation generating apparatus 110, the allowable time for emitting radiation in the general imaging mode such as the dynamic imaging mode is shorter than the allowable time for emitting radiation in the fluoroscopy mode. Instead of setting the allowable time, the number of allowable frames may be set. The number of allowable frames imaged by radiation generating apparatus 110 is fewer in the second mode than in the first mode.

Setting means 330 may cause limitation of the time for which radiation is emitted. In a case where the general imaging mode such as the dynamic imaging mode is selected, radiation generating apparatus 110 is not allowed to emit the radiation for the predetermined time or longer, whereas in a case where the fluoroscopy mode is selected, radiation generating apparatus 110 is allowed to emit the radiation for the predetermined time or longer.

Setting means 330 may limit the radiation dose by limiting the frame rate. The frame rate in the general imaging mode such as the dynamic imaging mode is lower than the frame rate in the fluoroscopy mode. The frame rate when radiation generating apparatus 110 performs imaging is lower in the second mode than in the first mode.

Setting means 330 may limit the radiation dose by limiting the pulse duty ratio. When radiation generating apparatus 110 emits a pulse of radiation, charges accumulate in radiation detection apparatus 120, and the radiation can be detected. Accordingly, the required pulse width of radiation is determined by the required image quality, the intensity of radiation to be detected, the capability of the radiation detection apparatus, and/or the like. The radiation emitted by radiation generating apparatus 110 has a lower pulse duty ratio in the general imaging mode such as the dynamic imaging mode than in the fluoroscopy mode.

Setting means 330 may limit the duty ratio by limiting the pulse width to limit the radiation dose. The radiation emitted by radiation generating apparatus 110 has a shorter pulse width in the general imaging mode such as the dynamic imaging mode than in the fluoroscopy mode.

Setting means 330 may limit the duty ratio by limiting the pulse interval to limit the radiation dose. The radiation emitted by radiation generating apparatus 110 has a longer pulse interval in the general imaging mode such as the dynamic imaging mode than in the fluoroscopy mode.

Setting means 330 may limit the radiation dose by limiting the intensity of radiation. The radiation emitted by radiation generating apparatus 110 has a lower intensity in the general imaging mode such as the dynamic imaging mode than in the fluoroscopy mode.

Setting means 330 may limit the intensity of radiation by controlling the tube of radiation generating apparatus 110 to limit the radiation dose.

Setting means 330 may limit the intensity of radiation by controlling the tube current-time product of the tube of radiation generating apparatus 110 to limit the radiation dose. Radiation generating apparatus 110 when radiation generating apparatus 110 emits radiation has a smaller tube current-time product in the general imaging mode such as the dynamic imaging mode than in the fluoroscopy mode.

Setting means 330 may limit the intensity of radiation by controlling the tube current of the tube of radiation generating apparatus 110 to limit the radiation dose. Radiation generating apparatus 110 when radiation generating apparatus 110 emits radiation has a smaller tube current of a tube in the general imaging mode such as the dynamic imaging mode than in the fluoroscopy mode.

Setting means 330 may limit the intensity of radiation by controlling the tube voltage of the tube of radiation generating apparatus 110 to limit the radiation dose. Radiation generating apparatus 110 when radiation generating apparatus 110 emits radiation has a lower tube voltage of a tube in the general imaging mode such as the dynamic imaging mode than in the fluoroscopy mode.

Setting means 330 may limit the radiation dose by shielding the radiation to be emitted from radiation generating apparatus 110 with a shielding object (filter). Radiation generating apparatus 110 includes a shielding means. In radiation generating apparatus 110, the shielding means shields the emitted radiation in the general imaging mode such as the dynamic imaging mode.

Setting means 330 may limit the radiation dose by reducing the intensity of the radiation to be emitted from generator 111 with a shielding object (filter). Radiation generating apparatus 110 includes a shielding means. In radiation generating apparatus 110, the shielding means shields the emitted radiation to reduce the intensity of the emitted radiation in the general imaging mode such as the dynamic imaging mode.

Setting means 330 may limit the radiation dose by limiting an area to be irradiated with the radiation that is emitted from generator 111. Radiation generating apparatus 110 includes a shielding means. In radiation generating apparatus 110, the shielding means shields a part of the emitted radiation to narrow an area to be irradiated with the emitted radiation in the general imaging mode such as the dynamic imaging mode.

Setting means 330 may limit the radiation dose by combining a plurality of the controls described above such that the allowable radiation dose is equal to or less than the allowable radiation dose corresponding to the set mode. Setting means 330 may limit the range of a setting value to be inputted by a user according to the allowable radiation dose. In a case where setting means 330 has determined that the allowable radiation dose is exceeded when radiation is emitted under a set condition (setting value), setting means 330 may cause input/output section 141 to send out a warning to prompt the user to change the setting value. Setting means 330 may output the warning to input/output section 141 to which the setting has been inputted, or may output the warning to input/output section 141 different from an input/output section to which the setting has been inputted. For example, a setting may be inputted through an external console, and the warning may be outputted to input/output section 141 of main body 140.

In a case where radiation emission is instructed by the emission instruction switch, setting means 330 may prompt the user to change a setting value. In prompting the user to change a setting value, the setting value which the user is prompted to change may be determined in consideration of set priorities. The priorities may be set according to users, may be set according to persons to be imaged, or may be set according to the imaging order for diseases to be diagnosed, and/or the like. For example, in a case where setting means 330 has determined that the allowable radiation dose is satisfied by changing one of the irradiation time and the frame rate, setting means 330 may prompt a user to change the setting value regarding the irradiation time based on the priorities. Setting means 330 may limit the range of a setting value to be inputted thereafter based on the allowable radiation dose and the setting value inputted by the user. In this case, setting means 330 preferably causes a user to input setting values in the order of priorities. In a case where radiation emission is instructed by the emission instruction switch, setting means 330 may prompt a user to change a setting value.

Setting means 330 may limit the radiation dose based on another index. For example, in a case where a still image is imaged, setting means 330 may determine based on the number of times of exposure for the still image whether the allowable radiation dose has been reached, and in a case where the allowable radiation dose has been emitted, setting means 330 may stop the radiation emission.

### [Flowchart]

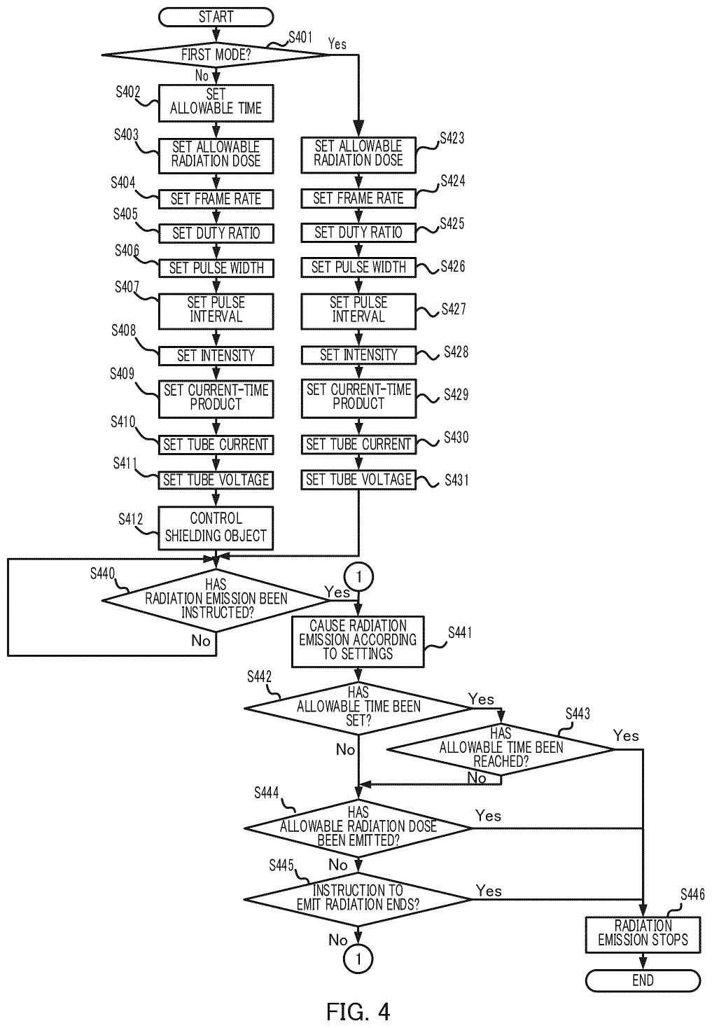

FIG. 4 is a diagram illustrating an example of a flowchart of processing performed by setting means 330.

Setting means 330 determines whether the mode is set to the first mode or the second mode (step S401). The first mode is the fluoroscopy mode. The second mode is the dynamic mode, but may be a mode for use as a general imaging apparatus.

In a case where the mode is not the first mode, that is, the mode is set to the second mode in step S401, setting means 330 sets the allowable time (step S402). The allowable time may not be set in the case of the first mode. After setting the allowable time, setting means 330 performs processing in step S403.

Setting means 330 sets the allowable radiation dose (step S403). In the second mode, the allowable radiation dose to be set does not exceed the radiation dose allowable in the laws and regulations. As the allowable radiation dose to be set, a radiation dose inputted through input/output section 141 may be set, or the allowable radiation dose may be automatically set in accordance with the laws and regulations. After setting the allowable radiation dose, setting means 330 performs the processing in step S404.

Setting means 330 sets the frame rate (step S404). After setting the frame rate, setting means 330 performs processing in step S405.

Setting means 330 sets the duty ratio (step S405). After setting the duty ratio, setting means 330 performs processing in step S406.

Setting means 330 sets the pulse width (step S406). After setting the pulse width, setting means 330 performs the processing in step S407.

Setting means 330 sets the pulse interval (step S407). After setting the pulse interval, setting means 330 performs the processing in step S408.

Setting means 330 sets the intensity of radiation (step S408). After setting the intensity of radiation, setting means 330 performs the processing in step S409.

Setting means 330 sets the current-time product (step S409). After setting the current-time product, setting means 330 performs the processing in step S410.

Setting means 330 sets the tube current of the tube (step S410). After setting the tube current, setting means 330 performs the processing in step S411.

Setting means 330 sets the tube voltage of the tube (step S411). After setting the tube voltage, setting means 330 performs the processing in step S412.

Setting means 330 controls the shielding object (step S412). The intensity of the emitted radiation may be reduced by the shielding object. The area to be irradiated with the emitted radiation may be limited by the shielding object. The area to be irradiated with radiation may be limited by collimator 112. After controlling the shielding object, setting means 330 performs the processing in step S440.

Setting means 330 may not perform a step or some steps among steps S403 to S412. The order of steps S402 to S412 may be changed.

In a case where the mode is set to the first mode in step S401, setting means 330 sets the allowable radiation dose (step S423). In a case where the mode is set to the first mode, the setting of the allowable radiation dose in step S423 may not be performed. In the first mode, the allowable radiation dose to be set does not exceed the radiation dose allowable in the laws and regulations. As the allowable radiation dose to be set, a radiation dose inputted through input/output section 141 may be set, or the allowable radiation dose may be automatically set in accordance with the laws and regulations. The allowable radiation dose to be set in step S423 may be larger than the allowable radiation dose set in step S403. After setting the allowable radiation dose, setting means 330 performs the processing in step S424.

Setting means 330 sets the frame rate (step S424). The frame rate to be set in step S424 may be higher than the frame rate to be set in step S404. After setting the frame rate, setting means 330 performs processing in step S425.

Setting means 330 sets the duty ratio (step S425). The duty ratio to be set in step S425 may be higher than the duty ratio to be set in step S405. After setting the duty ratio, setting means 330 performs processing in step S426.

Setting means 330 sets the pulse width (step S426). The pulse width set in step S426 may be longer than the pulse width set in step S406. After setting the pulse width, setting means 330 performs the processing in step S427.

Setting means 330 sets the pulse interval (step S427). The pulse interval to be set in step S427 may be longer than the pulse interval to be set in step S407. After setting the pulse interval, setting means 330 performs the processing in step S428.

Setting means 330 sets the intensity of radiation (step S428). The intensity of radiation to be set in step S428 may be larger than the intensity of the radiation to be set in step S408. After setting the intensity of radiation, setting means 330 performs the processing in step S429.

Setting means 330 sets the current-time product (step S429). The current-time product to be set in step S429 may be larger than the current-time product to be set in step S409. After setting the current-time product, setting means 330 performs the processing in step S430.

Setting means 330 sets the tube current of the tube (step S430). The tube current to be set in step S430 may be larger than the tube current to be set in step S410. After setting the tube current, setting means 330 performs the processing in step S431.

Setting means 330 sets the tube voltage of the tube (step S431). The tube voltage to be set in step S431 may be larger than the tube voltage to be set in step S411. After setting the tube voltage, setting means 330 performs the processing in step S440.

Setting means 330 may not perform a step or some steps among steps S423 to S432. The order of steps S423 to S431 may be changed.

When it is determined that the mode is the first mode, setting means 330 does not needs to set the allowable time and control the shielding object, but even in a case where it is determined that the mode is the first mode, setting means 330 may set the allowable time or control the shielding object.

Until step S431, the settings according to the first mode or the second mode end.

Setting means 330 determines whether radiation emission has been instructed (step S440).

In a case where setting means 330 has determined in step S440 that radiation emission has not been instructed, setting means 330 causes the flow to return to step S440 and waits for an instruction to emit radiation.

In a case where setting means 330 has determined in step S440 that radiation emission has been instructed, setting means 330 causes radiation generating apparatus 110 to emit radiation according to the settings (step S441). The radiation is emitted in a pulsed manner. When setting means 330 causes radiation generating apparatus 110 to emit radiation, setting means 330 performs the processing in step S442.

Setting means 330 determines whether the allowable time has been set (step S442).

In a case where setting means 330 has determined in step S442 that the allowable time has been set, setting means 330 determines whether the radiation time has reached the allowable time (step S443). Instead of determining whether the allowable time has been reached, setting means 330 may determine whether the number of allowable frames has been reached.

In a case where setting means 330 has determined in step S442 that the allowable time has not been set, or in a case where setting means 330 has determined in step S443 that the allowable time has not been reached, setting means 330 determines whether the allowable radiation dose has been emitted (step S444). The allowable radiation dose is defined by the laws and regulations, and the allowable radiation dose in a general imaging apparatus such as a dynamic imaging apparatus is smaller than the allowable radiation dose in a fluoroscopic apparatus.

In a case where setting means 330 has determined in step S444 that the allowable radiation dose has not been emitted, setting means 330 determines whether the instruction to emit radiation ends (step S445).

In a case where setting means 330 has determined in step S445 that the radiation emission instruction does not end, setting means 330 causes the flow to return to step S441 to cause radiation generating apparatus 110 to emit radiation according to the settings.

In a case where setting means 330 has determined in step S443 that the allowable time has been reached, in a case where setting means 330 has determined in step S444 that the allowable radiation dose has been emitted, or in a case where setting means 330 has determined in step S445 that the instruction to emit radiation ends, setting means 330 causes the radiation emission to stop (step S446).

The flowchart can also be executed by repeating only the emission of radiation from step S440 after the settings end.

### <Variations>

Setting means 330 desirably has a function to limit the settings of the fluoroscopy mode. Setting means 330 desirably performs user authentication and allows only the authenticated user to perform the settings of the fluoroscopy mode. The user to be authenticated is a user, such as a doctor and a radiology technician, who has been authorized by the hospital. The authentication can be performed using well-known authentication means such as an ID or an account, and a password, a staff certificate, and/or biometric authentication. Therefore, in a case where setting means 330 has determined in step S401 that the mode is set to the first mode, setting means 330 desirably performs user authentication before performing the processing in step S423. In a case where authentication as an authorized user has been performed, setting means 330 may execute the processing in step S423 and the subsequent steps. In a case where authentication as an authorized user has not been performed, setting means 330 may perform error processing and stop the processing in step S423 and the subsequent steps.

Setting means 330 may perform user authentication before the processing in step S401. In a case where authentication as an authorized user has been performed, setting means 330 may configure such that all the steps can be executed, whereas in a case where authentication as an authorized user has not been performed, setting means 330 may perform error processing by determining in step S401 that the mode is set to the first mode, and may stop the processing in step S423 and the subsequent steps.

In a case where the imaging order or the inspection order received from the RIS or the like is imaging or inspection using the fluoroscopy mode, setting means 330 may allow the settings of the first mode to be performed. Setting means 330 may perform settings based on the imaging order or the inspection order received from the RIS. The settings performed by setting means 330 based on the imaging order or the examination order received from the RIS may be changed by input/output section 141.

The fluoroscopic apparatus is required be used in a restricted area under the laws and regulations. Therefore, the fluoroscopy mode is desirably configured to be used only in a restricted area in terms of management in the hospital. Therefore, setting means 330 may be allowed to set the first mode in a case where setting means 330 has determined that the medical cart is present in a restricted area.

Instead of setting the allowable time and stopping the radiation emission according to the allowable time, setting means 330 may set the number of allowable frames and cause the radiation emission to stop according to the number of allowable frames. In radiation generating apparatus 110, the number of frames in the general imaging mode such as the dynamic imaging mode is smaller than the number of frames in the fluoroscopy mode.

In the fluoroscopy mode, the allowable time may be set. The allowable time in the fluoroscopy mode may be set longer than the allowable time in the general imaging mode such as the dynamic imaging mode. In this case, in the fluoroscopy mode, radiation emission that exceeds the allowable time in the general imaging mode such as the dynamic imaging mode is allowed, but radiation emission that exceeds the allowable time in the fluoroscopy mode is not allowed.

The radiation dose may be limited by the radiation dose to be emitted or by the radiation dose with which the human body is irradiated, for example, the effective dose. The effective dose is related to the radiation weighting factor and the tissue weighting factor.

Since the radiation weighting factor varies depending on the type of radiation to be emitted, for example, an α wave, a β wave, and a γ wave, the effective dose can be limited in consideration of the type of radiation to be emitted.

Since the tissue weighting factor varies depending on the region to be irradiated, the effective dose can be limited in consideration of the region to be irradiated.

Therefore, setting means 330 may set the type of radiation and/or the region to be irradiated.

According to the above embodiment, the radiation dose when used in the dynamic imaging mode is limited, and thus, for example, medical cart 100 can be brought into a general hospital room for dynamic imaging. Therefore, it is possible to perform dynamic imaging while reducing the burden on the patient and a healthcare professional.

Although an embodiment has been described above with reference to the drawings, the present disclosure is not limited to such an example. It is obvious that a person skilled in the art can conceive of various change examples or modification examples within the scope described in the claims. It is to be understood that such changes or modifications also belong to the technical scope of the present disclosure. Furthermore, the constituent elements in the embodiment may be combined as appropriate without departing from the spirit of the present disclosure.

(1) A mobile radiographic imaging system in an embodiment of the present disclosure includes: a radiation generating apparatus that emits radiation; and a hardware processor capable of setting the radiation generating apparatus to a plurality of modes including a first mode, in which the radiation generating apparatus performs moving image imaging with an allowable radiation dose, and a second mode, in which the radiation generating apparatus performs the moving image imaging with an allowable radiation dose smaller than the allowable radiation dose in the first mode.

(2) In the mobile radiographic imaging system in an embodiment of the present disclosure, in the radiography system of (1), an allowable time for the radiation generating apparatus to emit the radiation is shorter in the second mode than in the first mode.

(3) In the mobile radiographic imaging system in an embodiment of the present disclosure, in the radiography system of (1) or (2), in a case where the second mode is selected, the radiation generation apparatus is not allowed to emit the radiation exceeding a predetermined time.

(4) In the mobile radiographic imaging system in an embodiment of the present disclosure, in the radiography system of (3), in a case where the first mode is selected, the radiation generation apparatus is allowed to emit the radiation for the predetermined time or longer.

(5) In the mobile radiographic imaging system in an embodiment of the present disclosure, in the radiography system of any one of (1) to (4), the radiation generating apparatus stops emitting the radiation based on one of the allowable radiation doses.

(6) In the movable radiography system in an embodiment of the present disclosure, in the radiography system of any one of (1) to (5), a number of allowable frames to be imaged by the radiation generation apparatus is smaller in the second mode than in the first mode.

(7) In the mobile radiographic imaging system in an embodiment of the present disclosure, i n the radiography system of any one of (1) to (6), a frame rate when the radiation generating apparatus performs the moving image imaging is lower in the second mode than in the first mode.

(8) In the mobile radiographic imaging system in an embodiment of the present disclosure, in the radiography system of any one of (1) to (7), the radiation generating apparatus emits the radiation which is pulsed radiation, and the radiation emitted by the radiation generating apparatus has a lower pulse duty ratio in the second mode than in the first mode.

(9) In the mobile radiographic imaging system in an embodiment of the present disclosure, in the radiography system of any one of (1) to (8), the radiation generating apparatus emits the radiation that is pulsed radiation, and the radiation emitted by the radiation generating apparatus has a shorter pulse width in the second mode than in the first mode.

(10) In the mobile radiographic imaging system in an embodiment of the present disclosure, in the radiography system of any one of (1) to (9), the radiation generating apparatus emits the radiation that is pulsed radiation, and the radiation emitted by the radiation generating apparatus has a longer pulse interval in the second mode than in the first mode.

(11) In the mobile radiographic imaging system in an embodiment of the present disclosure, in the radiography system of any one of (1) to (10), the radiation emitted by the radiation generation apparatus has a lower intensity in the second mode than in the first mode.

(12) In the mobile radiographic imaging system in an embodiment of the present disclosure, in the radiography system of any one of (1) to (11), the radiation generating apparatus when the radiation generating apparatus emits the radiation has a smaller tube current-time product in the second mode than in the first mode.

(13) In the mobile radiographic imaging system in an embodiment of the present disclosure, in the radiography system of any one of (1) to (12), the radiation generating apparatus when the radiation generating apparatus emits the radiation has a smaller tube current of a tube in the second mode than in the first mode.

(14) In the mobile radiographic imaging system in an embodiment of the present disclosure, in the radiography system of any one of (1) to (13), the radiation generating apparatus when the radiation generating apparatus emits the radiation has a lower tube voltage of a tube in the second mode than in the first mode.

(15) In the mobile radiographic imaging system in an embodiment of the present disclosure, in the radiography system of any one of (1) to (14), the radiation generating apparatus includes a shielding means, and the shielding means shields the radiation emitted in the second mode.

(16) In the mobile radiographic imaging system in an embodiment of the present disclosure, in the radiography system of any one of (1) to (15), the radiation generating apparatus includes a shielding means, and the shielding means reduces intensity of the radiation emitted in the second mode.

(17) In the mobile radiographic imaging system in an embodiment of the present disclosure, in the radiography system of any one of (1) to (16), the radiation generating apparatus includes a shielding means, and the shielding means narrows an area to be irradiated with the radiation emitted in the second mode.

(18) In the mobile radiographic imaging system in an embodiment of the present disclosure, in the radiography system of any one of (1) to (17), the first mode is a fluoroscopy mode, and the second mode is a dynamic imaging mode.

(19) In the mobile radiographic imaging system in an embodiment of the present disclosure, in the radiography system of any one of (1) to (18), the allowable radiation doses are effective doses.

(20) In the mobile radiographic imaging system in an embodiment of the present disclosure, in the radiography system of any one of (1) to (19), the hardware processor performs control such that a radiation dose is limited to be equal to or less than the allowable radiation dose in a set imaging mode, where the set imaging mode is one of the first mode and the second mode.

(21) In the mobile radiographic imaging system in an embodiment of the present disclosure, in the radiography system of any one of (1) to (20), further comprising a display means that displays a moving image imaged by the moving image imaging based on a set imaging mode, where the set imaging mode is one of the first mode and the second mode, and in a case where the second mode is set by the hardware processor, real-time display of the moving image by the display means is limited.

(22) In the mobile radiographic imaging system in an embodiment of the present disclosure, in the radiography system of any one of (1) to (21), the hardware processor has a function to limit at least a setting of the first mode.

(23) In the mobile radiographic imaging system in an embodiment of the present disclosure, in the radiography system of any one of (1) to (22), in a case where the second mode is set, the hardware processor causes the radiation generating apparatus to stop emitting the radiation when an imaging time is equal to or longer than a predetermined time.

(24) In the mobile radiographic imaging system in an embodiment of the present disclosure, in the radiography system of any one of (1) to (23), further comprising an inputter that inputs a setting of the radiation generating apparatus to the hardware processor, wherein in a case where the second mode is set by the hardware processor, the inputter is prohibited from inputting a setting for an allowable radiation dose that exceeds an allowable radiation dose in the second mode.

### INDUSTRIAL APPLICABILITY

The present disclosure is useful for a radiography apparatus.

Although embodiments of the present invention have been described and illustrated in detail, the disclosed embodiments are made for purpose of illustration and example only and not limitation. The scope of the present invention should be interpreted by terms of the appended claims.

What is claimed is:

1. A mobile radiographic imaging system, comprising:
a radiation generating apparatus that emits radiation; and
a hardware processor capable of setting the radiation generating apparatus to one of a plurality of modes including a first mode and a second mode, the first mode being a mode in which the radiation generating apparatus performs moving image imaging with an allowable radiation dose, the second mode being a mode in which the radiation generating apparatus performs the moving image imaging with an allowable radiation dose smaller than the allowable radiation dose in the first mode.

2. The mobile radiographic imaging system according to claim 1, wherein
an allowable time for the radiation generating apparatus to emit the radiation is shorter in the second mode than in the first mode.

3. The mobile radiographic imaging system according to claim 1, wherein
in a case where the second mode is selected, the radiation generation apparatus is not allowed to emit the radiation for the predetermined time or longer.

4. The mobile radiographic imaging system according to claim 3, wherein
in a case where the first mode is selected, the radiation generation apparatus is allowed to emit the radiation for the predetermined time or longer.

5. The mobile radiographic imaging system according to claim 1, wherein
the radiation generating apparatus stops emitting the radiation based on one of the allowable radiation doses.

6. The mobile radiographic imaging system according to claim 1, wherein
a number of allowable frames to be imaged by the radiation generation apparatus is smaller in the second mode than in the first mode.

7. The mobile radiographic imaging system according to claim 1, wherein
a frame rate when the radiation generating apparatus performs the moving image imaging is lower in the second mode than in the first mode.

8. The mobile radiographic imaging system according to claim 1, wherein:
the radiation generating apparatus emits the radiation which is pulsed radiation, and
the radiation emitted by the radiation generating apparatus has a lower pulse duty ratio in the second mode than in the first mode.

9. The mobile radiographic imaging system according to claim 1, wherein:
the radiation generating apparatus emits the radiation that is pulsed radiation, and
the radiation emitted by the radiation generating apparatus has a shorter pulse width in the second mode than in the first mode.

10. The mobile radiographic imaging system according to claim 1, wherein:
the radiation generating apparatus emits the radiation that is pulsed radiation, and
the radiation emitted by the radiation generating apparatus has a longer pulse interval in the second mode than in the first mode.

11. The mobile radiographic imaging system according to claim 1, wherein
the radiation emitted by the radiation generation apparatus has a lower intensity in the second mode than in the first mode.

12. The mobile radiographic imaging system according to claim 1, wherein
the radiation generating apparatus when the radiation generating apparatus emits the radiation has a smaller tube current-time product in the second mode than in the first mode.

13. The mobile radiographic imaging system according to claim 1, wherein
the radiation generating apparatus when the radiation generating apparatus emits the radiation has a smaller tube current of a tube in the second mode than in the first mode.

14. The mobile radiographic imaging system according to claim 1, wherein
the radiation generating apparatus when the radiation generating apparatus emits the radiation has a lower tube voltage of a tube in the second mode than in the first mode.

15. The mobile radiographic imaging system according to claim 1, wherein:
the radiation generating apparatus includes a shield, and
the shield shields the radiation emitted in the second mode.

16. The mobile radiographic imaging system according to claim 1, wherein:
the radiation generating apparatus includes a shield, and
the shield reduces intensity of the radiation emitted in the second mode.

17. The mobile radiographic imaging system according to claim 1, wherein:
the radiation generating apparatus includes a shield, and
the shield narrows an area to be irradiated with the radiation emitted in the second mode.

18. The mobile radiographic imaging system according to claim 1, wherein:
the first mode is a fluoroscopy mode, and
the second mode is a dynamic imaging mode.

19. The mobile radiographic imaging system according to claim 1, wherein
the allowable radiation doses are effective doses.

20. The mobile radiographic imaging system according to claim 1, wherein
the hardware processor performs control such that a radiation dose is limited to be equal to or less than the allowable radiation dose in a set imaging mode, the set imaging mode being one of the first mode and the second mode.

21. The mobile radiographic imaging system according to claim 1, further comprising
a display that displays a moving image imaged by the moving image imaging based on a set imaging mode, the set imaging mode being one of the first mode and the second mode, wherein
in a case where the second mode is set by the hardware processor, real-time display of the moving image by the display is limited.

22. The mobile radiographic imaging system according to claim 1, wherein
the hardware processor has a function to limit at least a setting of the first mode.

23. The mobile radiographic imaging system according to claim 1, wherein
in a case where the second mode is set, the hardware processor causes the radiation generating apparatus to stop emitting the radiation when an imaging time is equal to or longer than a predetermined time.

24. The mobile radiographic imaging system according to claim 1, further comprising
an inputter that inputs a setting of the radiation generating apparatus to the hardware processor, wherein
in a case where the second mode is set by the hardware processor, the inputter is prohibited from inputting a setting for an allowable radiation dose that exceeds an allowable radiation dose in the second mode.
